(19) United States
(12) Patent Application Publication
(10) Pub. No.: US 2019/0133482 A1
(43) Pub. Date: May 9, 2019

(54) METHOD AND SYSTEM FOR FATIGUE DETERMINATION

(71) Applicant: SWEDISH ADRENALINE AB, Halmstad (SE)

(72) Inventors: Per-Arne Viberg, Halmstad (SE); Petras RAZANSKAS, Kaunas (LT); Antanas Verikas, Halmstad (SE)

(73) Assignee: SWEDISH ADRENALINE AB, Halmstad (SE)

(21) Appl. No.: 15/772,639

(22) PCT Filed: Nov. 4, 2016
(86) PCT No.: PCT/EP2016/076633

§ 371 Date: May 1, 2018

(30) Foreign Application Priority Data
Nov. 5, 2015 (SE) 1551432-6

(51) Int. Cl. A61B 5/0488; A61B 5/04; A61B 5/22; A61B 5/00

(52) U.S. Cl. CPC A61B 5/0488; A61B 5/04012; A61B 2503/10; A61B 5/4884; A61B 5/221

(57) ABSTRACT

A technology is disclosed for indicating the fatigue of a person. It comprises: obtaining (102) a plurality of simultaneously recorded signals, wherein each signal is associated with a muscle of the person and indicates the electrical activity of the muscles in the time domain. The technology further comprises: determining (104) a set of points in time from each signal, wherein each point in time indicates a change in the state of the associated muscle, and determining (106) the measure indicating the fatigue of the person based on the set of points in time from the plurality of simultaneously recorded signals.

### TECHNICAL FIELD

The invention relates to the determining of a measure or indication of fatigue from signals indicating the electrical activity of muscles.

### BACKGROUND ART

For professional cyclists, as well as dedicated amateurs, fatiguing cycling sessions are important for building up muscle power, raising lactate threshold, and improving oxygen uptake. However, excessive fatigue with insufficient recovery time can result in over-training, and increased susceptibility to injuries, which can have a negative long term impact on the career and health of an athlete or cycling enthusiast. This is also true for other sports and other forms of exercise than cycling, such as weight lifting, running, swimming, and rowing. Therefore, it is desirable to enable appropriate feedback to the user so that the training regimen can be adjusted appropriately to avoid the aforementioned risks.

Surface electromyography (sEMG) is an available technique for non-invasive acquisition of data about muscle activation. The classic way of estimating muscle fatigue in static exercises with relatively high loads is by calculating the median power frequency (MPF) of the sEMG signal in overlapping windows of a Short-Time Fourier Transform, and observing its decrease over time. However, in the case of dynamic exercise, the applicability and accuracy of this technique has been called into question.

Moreover, calculating MPF requires the use of Fourier transforms, which is a relatively expensive operation in terms of computation and power consumption. Due to the expensive operation, the MPF technology is typically limited to stationary systems or requires large batteries, thus limiting the use to activities that can be performed in close proximity to the system or in a stationary setting, such as cycling on stationary bicycle.

### SUMMARY

It is an object of the invention to at least partly overcome one or more of the above-identified limitations of the prior art. In particular, it is an object to improve the accuracy of fatigue determining in dynamic exercises. It is also an object to provide a technology for determining fatigue that is easy to carry. It is a further object to provide a robust technology for determining fatigue that requires a minimal adaption to a user.

### GENERAL DESCRIPTION

To solve the above objects, a method is provided for determining a measure indicating the fatigue of a person. The method comprises: obtaining a plurality of simultaneously recorded signals, wherein each signal is associated with a muscle of the person and indicates the electrical activity of the muscles in the time domain. The method further comprises: determining a set of points in time from each signal, wherein each point in time indicates a change in the state of the associated muscle, and determining the measure indicating the fatigue of the person based on, or based at least in part on, the sets of points in time for the plurality of simultaneously recorded signals.

The set of points in time may be determined in the time domain of each signal. Thus, the analysis is performed in the time domain, which means that less resources in terms of processing and power is required than for methods depending on a Fourier transforms. Thus, the method can be implemented in systems that are smaller and lighter, thus being easier to carry. The method also allows for an improved accuracy in fatigue determining for dynamic exercises.

Here, fatigue is understood to encompass one or more of muscle fatigue, physical fatigue, metabolic fatigue, blood lactate concentration, oxygen uptake, and one or more physiological parameters affected by physical exercise or activity. The measure indicating the fatigue is understood to encompass an indication of the fatigue. Each of the plurality of simultaneously recorded signals may be a surface electromyography signal. The change in the state of the associated muscle may be a change of the electrical activity of the associated muscle. The change in the state of the associated muscle may correspond to a change between contraction and relaxation of the associated muscle, or an activation or deactivation of a muscle. A measure may be represented by a number. That each signal is associated with a muscle is understood to encompass that each signal originates from the muscle.

Each set of points in time may comprise activation times, wherein each activation time indicates the start of an active state of the associated muscle. The start of an active state may correspond to the start of an electrical activity of the associated muscle, or an activation or contraction of the associated muscle. Each set of points in time may comprise deactivation times, wherein each deactivation time indicates the start of an inactive state of the associated muscle. The start of an inactive state may correspond to the end of an electrical activity of the associated muscle, or a deactivation or relaxation of the associated muscle. Each set of points in time may be composed of the activation times and the deactivation times.

Determining the measure indicating the fatigue of the person may further be based on a model depending on a plurality of input parameters, wherein each input parameter is derived from the set of points in time determined for a signal of the plurality of simultaneously recorded signals. The model may be based on one or more machine learning methods. The machine learning method may be based on or derived from a random forest method.

Determining the measure indicating the fatigue may comprise determining the measure based on one or more mean values, wherein each mean value is determined as a mean based on the amplitude over periods of time defined by the points in time of a signal of the plurality of simultaneously recorded signals. Alternatively, determining the measure indicating the fatigue may comprise determining the measure based on one or more mean root-mean square values, wherein each mean root-mean square values is determined from a signal and the points in time of the signal. Additionally or alternatively, determining the measure indicating the fatigue may comprise determining the measure based on one or more mean phase shifts, wherein each mean phase shift is determined between points in time of two different signals. Additionally or alternatively, determining the measure indicating the fatigue may comprise determining the measure based one or more mean active-time intervals, wherein each mean active-time interval is determined from time intervals between points in time of a signal.

The method may further comprise: determining from each signal an indication of the change in activity, or muscle activity, over time of the associated muscle. The set of points in time for the associated muscle may be derived from the indication of the change in activity over time.

The step of determining from each signal an indication of the change in activity over time may comprise: forming a time sequence of ratios, wherein each ratio is formed by a first sum over a second sum, and the first sum is a sum of absolute values of the signal over a first window in time, and the second sum is a sum of absolute values of the signal over a second window in time, and determining the indication of the change in activity based on the change in time of the sequence of ratios. The first window and the second window may be shifted, adjacent, and or consecutive in time. Additionally or alternatively, the first window and the second window may be overlapping and/or of equal length.

Each point in time, or activation time or deactivation time, may be determined as the time at a local extrema of the indication of the change in activity. Each activation time may be a local maxima and each deactivation time may be a local minima, or vice versa. The nature of the extrema depends on the order of first window and second window.

The step of determining the measure indicating the fatigue may comprise: determining one or more mean root-mean square values, wherein each of these root-mean square values is determined as the mean of a plurality of root-mean square values determined from a single signal of the plurality of simultaneously recorded signals. Each root-mean square value may be an input parameter of the abovementioned model. Additionally, each of these root-mean square values may be determined based on the root-mean square of the amplitude for a time period of the single signal, wherein each time period is located between an activation time and a directly following, or consecutive, activation time of the single signal. Additionally, the measure indicating the fatigue may further be determined, or at least in part determined, based on the determined one or more mean root-mean square values.

Each time period may further be located between an activation time and a directly following, or consecutive, deactivation time of the same signal. The two points in time may correspond to the end points of the period. Alternatively, one of the points in time may correspond to an end point of the period.

The one or more mean root-mean square values may comprise a mean root-mean square value determined from a signal associated with a first muscle controlling a limb and another mean root-mean square value determined from a signal associated with another muscle controlling another limb. These features are advantageous for indicating blood lactate concentration. The one or more mean root-mean square values may comprise a mean root-mean square value determined from a signal associated with a first muscle controlling a limb and another mean root-mean square value determined from a signal associated with another muscle controlling the same limb. These features are advantageous for indicating oxygen uptake.

A muscle controlling a limb is here and throughout these specifications understood to encompass muscles connected only to the limb itself, and muscles connected to the limb and to other parts of the body of the person, such as the torso. For example, the first muscle may be the gluteus maximus and the limb may be the leg on the same side of the body, or the first muscle may be the pectoralis major and the limb may be the arm on the same side of the body.

The one or more mean root-mean square values may comprise a mean root-mean square value determined from a signal associated with a rectus femoris. These features are particularly advantageous for indicating blood lactate concentration and oxygen uptake.

The one or more mean root-mean square values may comprise a mean root-mean square value determined from a signal associated with a semitendinosus. These features are particularly advantageous for indicating blood lactate concentration.

The one or more mean root-mean square values may comprise a mean root-mean square value determined from a signal associated with a vastus lateralis. These features are particularly advantageous for indicating oxygen uptake.

Determining the measure indicating the fatigue may comprise: determining one or more mean phase shifts, wherein each of these mean phase shifts is determined as the mean of a plurality of phase shifts determined from a pair of signals of the plurality of simultaneously recorded signals, and each of these phase shifts is determined based on a first time interval between an activation time, or deactivation time, of a signal of the pair of signals and an directly following, or consecutive, activation time, or deactivation time, of the other signal of the pair of signals. Additionally, the measure indicating the fatigue may further be determined, or at least in part determined, based on the determined one or more mean phase shifts. Each mean phase shift may be an input parameter of the abovementioned model.

The pair of signals may be associated with a pair of muscles controlling the same limb. The one or more mean phase shifts may comprises a mean phase shift determined from a first pair of signals associated with a first pair of muscles controlling a limb and another mean phase shifts determined from a second pair of signals associated with a second pair of muscles controlling the same limb. Additionally or alternatively, the one or more mean phase shifts may comprise a mean phase shift determined from a first pair of signals associated with a first pair of muscles controlling a limb and another mean phase shifts determined from a second pair of signals associated with a second pair of muscles controlling another limb. These features are particularly advantageous for indicating blood lactate concentration and oxygen uptake.

A pair of muscles, including first pair and second pair, may be formed by the rectus femoris and the vastus lateralis. These features are particularly advantageous for indicating blood lactate concentration and oxygen uptake.

A pair of muscles, including first pair and second pair, may be formed by the vastus lateralis and the semitendinosus. Additionally or alternatively, a pair of muscles, including first pair and second pair, controlling the same limb may be formed by the rectus femoris and the semitendinosus. These features are particularly advantageous for indicating blood lactate concentration.

A pair of signals may be associated with a pair of muscles controlling different limbs. The different limbs may be of the same kind. This means that the limbs are either the arms or the legs of the person. The pair of muscles may be formed by the rectus femoris of the right leg and the rectus femoris of the left leg. These features are particularly advantageous for indicating oxygen uptake.

For each mean phase shift, the plurality of phase shifts may further be determined from a first additional signal of the plurality of simultaneously recorded signals. Additionally, each first time interval may be normalized by a second time interval between two consecutive activation times of the first additional signal and covering at least a portion of the first time interval. Covering at least a portion of an interval is here understood to encompass covering the complete interval including or excluding the end points, covering a portion of the interval and one end point, and covering a portion within the interval.

The first additional signal and the pair of signals may be associated with muscles on the same limb. The first additional signal and one of the signals of the pair of signals may be associated with muscles on the same limb. The first additional signal is associated with the rectus femoris.

Determining the measure indicating the fatigue may comprise: determining one or more mean active-time intervals. Each of these mean active-time intervals may be determined as the mean of a plurality of active-time intervals determined from a single signal of the plurality of simultaneously recorded signals, and each of these active-time intervals may be determined based on a third time interval between an activation time and a directly following, or consecutive, deactivation time. Additionally, the measure indicating the fatigue may further be determined, or at least in part determined, based on the determined one or more mean active-time intervals. Each mean active-time interval may be an input parameter of the abovementioned model.

The one or more mean active-time intervals comprises a mean active-time intervals determined from a signal associated with a first muscle controlling a limb and another mean active-time intervals determined from a signal associated with another muscle controlling another limb. The one or more mean active-time intervals may comprise a mean active-time intervals determined from a signal associated with a rectus femoris. Additionally or alternatively, the one or more mean active-time intervals may comprise a mean active-time interval determined from a signal associated with a vastus lateralis. These features are particularly advantageous for indicating blood lactate concentration.

For each mean active-time interval, the plurality of active-time intervals may further be determined from a second additional signal of the plurality of simultaneously recorded signals. Additionally, each third time interval may be normalized by a fourth time interval between two consecutive activation times of the second additional signal and covering at least a portion of the third time interval. Covering at least a portion of an interval is here understood to encompass covering the complete interval including or excluding the end points, covering a portion of the interval and one end point, and covering a portion within the interval. The second additional signal and the single signal may be associated with different muscles on the same limb. Alternatively, the second additional signal and the single signal may be associated with the same type of muscle on different limbs. The second additional signal may be associated with a rectus femoris. These features are advantageous for indicating blood lactate concentrations.

To solve the above objects, a system is also provided for determining a measure indicating the fatigue of a person. The system comprises one or more detectors for simultaneously recording signals indicating the electrical muscle activity in the time domain, a processor for executing program instructions, a non-volatile memory comprising program instructions. The program instructions are configured to, when executed by the processor, cause the system to: obtain a plurality of simultaneously recorded signals from the one or more detectors, wherein each signal is associated with a muscle of the person and indicates the electrical activity of the muscles in the time domain. The program instructions are further configured to cause the system to: determine a set of points in time from each signal, wherein each point in time indicates a change in the state of the associated muscle, and to determine the measure indicating the fatigue of the person based on the set of points in time from the plurality of simultaneously recorded signals.

The system may further comprise a display, and the program instructions may further be configured to, when executed by the processor, cause the system to: indicate the measure indicating the fatigue of the person on the display. The program instructions may further be configured to, when executed by the processor, cause the system to perform any of the steps or to comprise any of the features of the method described above.

The detectors may be skin contact electrodes. The skin contact electrodes may be embedded in a textile, such as textile of cycling shorts. This has the effect that data acquisition is easier and more accessible. The system may comprise a smartphone. The smartphone may comprise the processor and the non-volatile memory. The detectors may be configured to communicate with the smartphone by wire or wirelessly. The display may be the display of the smartphone or a peripheral device to the smartphone, such as a smartwatch. These features have the effect that real-time data evaluation and dynamic feedback is readily available to the user.

To solve the above objects, a computer program product is also provided for use in a system for determining a measure indicating the fatigue of a person. The system comprises one or more detectors for simultaneously recording signals indicating the electrical muscle activity in the time domain, and a processor for executing program instructions. The computer program product comprises program instructions configured to, when executed by the processor, cause the system to: obtain a plurality of simultaneously recorded signals from the one or more detectors, wherein each signal is associated with a muscle of the person and indicates the electrical activity of the muscles in the time domain. The program instructions are further configured to cause the system to: determine a set of points in time from each signal, wherein each point in time indicates a change in the state of the associated muscle, and determine the measure indicating the fatigue of the person based on the set of points in time from the plurality of simultaneously recorded signals. The program instructions may further be configured to, when executed by the processor, cause the system to perform any of the steps or to comprise any of the features of the method described above. The computer program product may be stored in a non-volatile memory.

### BRIEF DESCRIPTION OF THE DRAWINGS

Embodiments of the invention are described, by way of example, with reference to the accompanying drawings, in which:

FIG. 1 is a schematic view of a system for determining a measure indicating the fatigue of a person,

FIG. 2 is a flow chart illustrating a method for determining a measure indicating the fatigue of a person,

FIGS. 3-6 are flow chart illustrating further details of the method of FIG. 2, and

FIGS. 7-12 are diagrams and tables supporting a proof-of-concept.

### DETAILED DESCRIPTION

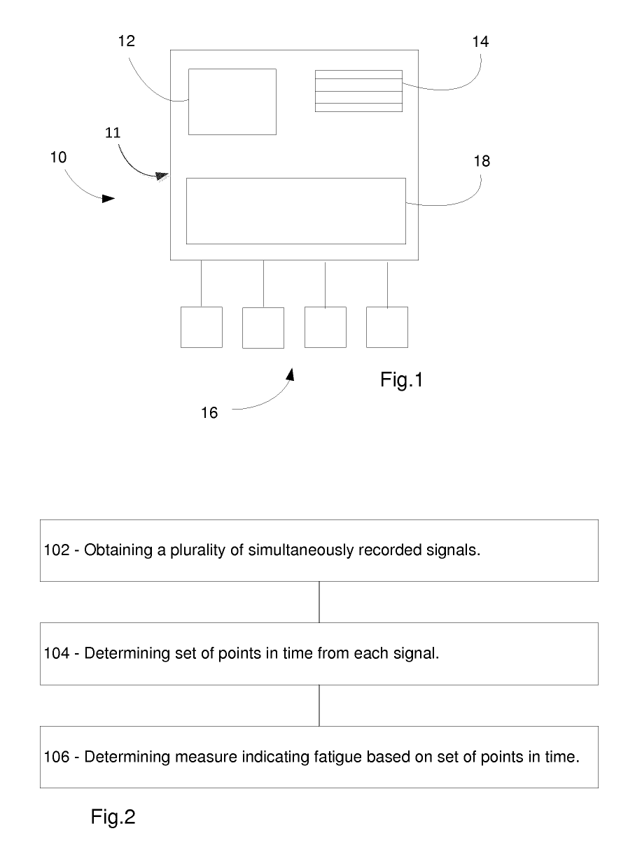

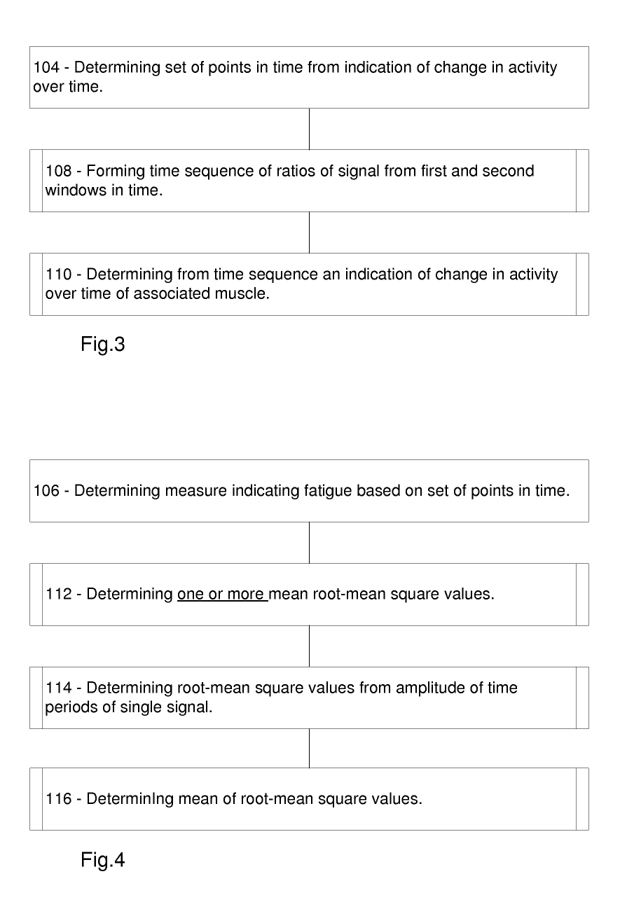

A schematic view of a system 10 for determining a measure indicating fatigue, or more precisely, the blood lactate concentration and the oxygen uptake, of a person is shown in FIG. 1. The system 10 comprises one or more detectors 16 that can simultaneously record signals indicating the electrical muscle activity in the time domain when placed on a person. The detectors 16 are surface electromyography electrodes configured to be placed on and attached to the skin. The system also has a processor 12 that can execute program instructions and a non-volatile memory 14 in which program instructions are stored. When executed by the processor 12, the program instructions causes the system to perform a method, which is further described in relation to FIGS. 2-6. The program instructions are stored in the non-volatile memory 14 as a computer program product.

The system further has a display 18, and the program instructions are further configured to cause the system 10 to indicate the measure indicating the fatigue on the display 18. The processor, non-volatile memory, and display forms parts of a smartphone 11, and the detectors 16 communicate with the smartphone 11 by wire.

A flow chart is shown in FIG. 2 illustrating a method for determining a measure indicating the blood lactate concentration and the oxygen uptake of a person. In the method, a plurality of simultaneously recorded signals is obtained 102 by the detectors 16. Each of the signals is associated with a muscle of the person and indicates the electrical activity of the muscles in the time domain. A set of points in time are then determined 104 from each signal, and each point in time indicates a change in the state of the associated muscle. The measure indicating the fatigue of the person is then determined 106 from the set of points in time. The set of points in time includes activation times and deactivation times. Each activation time indicates the start of an active state of the associated muscle, and each deactivation time indicates the start of an inactive state of the associated muscle.

The step of determining 106 the measure indicating the fatigue of the person is based on a model with several input parameters, where each parameter is based the set of points in time from one signal. The model in question is based on a random tree model. A choice of input parameters of the model is described in the proof-of-concept below.

An indication of the change in activity, or muscle activity, over time of the associated muscle is determined 108 from each signal. For each signal, this includes the forming 108 of a time sequence of ratios. Each ratio, or ratio of variability, is formed by a first sum over a second sum. The first sum is a sum of absolute values of the signal over a first window in time. The second sum is a sum of absolute values of the signal over a second window in time. The first window and the second window are shifted in time and of equal length. The indication of the change in activity is then determined 110 based on the change in time of the sequence of ratios, and the set of points in time of the signal relating to the associated muscle are then derived 104 from the indication of the change in activity over time.

Each activation time and deactivation time is determined as the time at a local extrema of the indication of the change in activity. More precisely, each activation time is a local maxima and each deactivation time is a local minima.

The step of determining 106 the measure indicating the fatigue includes the determining 112 of one or more mean root-mean square values, as is illustrated in FIG. 4. Each of these root-mean square values is determined 116 as the mean of a plurality of root-mean square values determined from a single signal. Each of these root-mean square values is determined 114 as the root-mean square of the amplitude for a time period of the single signal. Here, each time period is located between an activation time and a directly following deactivation time of the single signal. Each mean root-mean square value is an input parameter in the abovementioned model, which means that the measure indicating the fatigue is then determined 106 based on the determined one or more mean root-mean square values.

For determining a measure indicating the blood lactate concentration, the one or more mean root-mean square values includes a mean root-mean square value determined from a signal associated with a rectus femoris of one leg and a mean root-mean square value determined from a signal associated with a semitendinosus of the other leg. This means that the one or more mean root-mean square values includes a mean root-mean square value that is determined from a signal associated with a first muscle controlling a limb and another mean root-mean square value determined from a signal associated with another muscle controlling another limb.

For determining a measure indicating the oxygen uptake, the one or more mean root-mean square values includes a mean root-mean square value determined from a signal associated with a rectus femoris and a mean root-mean square value determined from a signal associated with a vastus lateralis. This means that the one or more mean root-mean square values includes a mean root-mean square value that is determined from a signal associated with a first muscle controlling a limb and another mean root-mean square value determined from a signal associated with another muscle controlling the same limb.

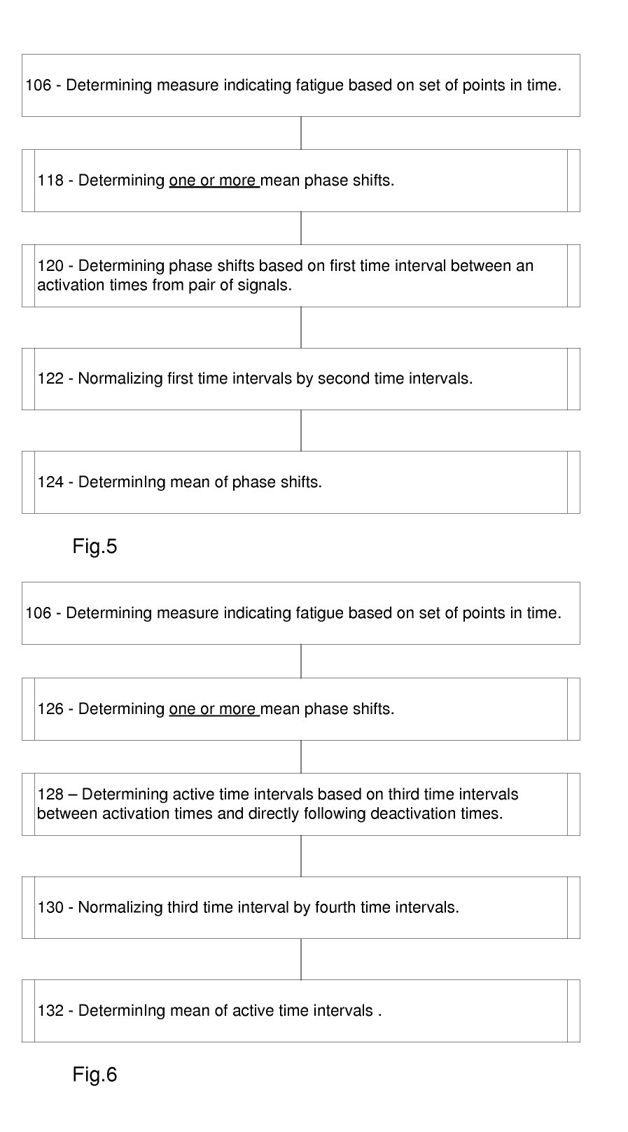

The step of determining 106 the measure indicating the fatigue further includes the determining 118 of one or more mean phase shifts, as is illustrated in FIG. 5. Each of these mean phase shifts is determined 124 as the mean of a plurality of phase shifts derived from a pair of signals and from a first additional signal of the plurality of simultaneously recorded signals. Each phase shifts is determined 120 as a first time interval between an activation time of a signal of the pair of signals and an directly following activation time of the other signal of the pair of signals. Each first time interval is further normalized 122 by a second time interval between two consecutive activation times of the first additional signal, where the second time interval covers a portion of the first time interval. Each mean phase shift is an input parameter in the abovementioned model, which means that the measure indicating the fatigue is determined 106 based on the determined one or more mean phase shifts.

When determining 106 the measure indicating the blood lactate concentration the pairs of signals are associated with pairs of muscles controlling the same limb, which in this case is a leg. The one or more mean phase shifts includes mean phase shifts determined from the signals associated with a pair of muscles formed by the rectus femoris and the vastus lateralis, and a pair of muscles formed by the vastus lateralis and the semitendinosus. This means that the one or more mean phase shifts includes a mean phase shift determined from a first pair of signals associated with a first pair of muscles controlling a limb and another mean phase shifts determined from a second pair of signals associated with a second pair of muscles controlling the same limb.

The one or more mean phase shifts further includes a mean phase shift determined from the signals associated with a pair of muscles formed the rectus femoris and the semitendinosus. This pair of muscles is located on one leg of the person, while the other pairs of muscles described above are located on the other leg of the person. This means that the one or more mean phase shifts comprises a mean phase shift determined from a first pair of signals associated with a first pair of muscles controlling a limb and another mean phase shifts determined from a second pair of signals associated with a second pair of muscles controlling another limb.

When determining 106 the measure indicating the oxygen uptake, the pairs of signals are associated with pairs of muscles controlling the limb, which in this case is a leg. The one or more mean phase shifts then includes a mean phase shift determined from the signals associated with a pair of muscles formed by the rectus femoris and the vastus lateralis. When determining 106 the measure indicating the oxygen uptake, the pairs of signals are also associated with pairs of muscles controlling different limbs of the same kind, which in this case are both legs. The one or more mean phase shifts then includes a mean phase shift determined from the signals associated with a pair of muscles formed by the rectus femoris of the right leg and the rectus femoris of the left leg.

The abovementioned first additional signal is associated with the rectus femoris on one of the legs. This means that the first additional signal and a pair of signals are associated with muscles on the same limb, but also that the first additional signal and one of the signals of a pair of signals are associated with muscles on the same limb.

The step of determining 106 the measure indicating the fatigue further includes the determining 126 of one or more mean active-time intervals, as is illustrated in FIG. 6. Each mean active-time interval is determined 132 as the mean of a plurality of active-time intervals derived from a single signal and a second additional signal of the plurality of simultaneously recorded signals. Each active-time interval is determined 128 based on a third time interval between an activation time and a directly following deactivation time normalized 130 by a fourth time interval between two consecutive activation times of the second additional signal that covers at least a portion of the third time interval. Each mean active-time interval is an input parameter in the abovementioned model, which means that measure indicating the fatigue is determined 106 based on the determined one or more mean active-time intervals.

When determining 106 the measure indicating the blood lactate concentration one or more mean active-time intervals includes a mean active-time interval determined from a signal associated with a rectus femoris on one leg and a mean active-time interval determined from a signal associated with a vastus lateralis on the other leg. This means that the one or more mean active-time intervals includes a mean active-time intervals determined from a signal associated with a first muscle controlling a limb and another mean active-time intervals determined from a signal associated with another muscle controlling another limb.

The second additional signal may be associated with the rectus femoris on the other leg than the abovementioned rectus femoris. This means that the second additional signal and the single signal are associated with different muscles on the same limb, and that the second additional signal and the single signal are associated with the same type of muscle on different limbs.

### Proof-of-Concept

An investigation has been performed showing that the proposed technology works. A data collection was first performed, in which data from 9 test subjects—5 male and 4 female, with the mean age of 35±12 years, mean weight of 71±11 kg, and mean height of 174±11 cm—were collected for the purpose of this research. The testing protocol followed three phases, defined by the load of a cyclist:

1. cycling at 50% of cyclist's VO₂ threshold (VO₂ₘₐₓ; power level, after which oxygen uptake no longer increases; measured prior to the experiments) for 6 minutes;

2. cycling at 90-95% of VO₂ₘₐₓ until the cyclist was no longer able to continue the task; and

3. after a break of no longer than 15 seconds, continue cycling at 50% of VO₂ₘₐₓ for another 6 minutes.

A steady cadence of 90 to 100 rotations per minute was maintained throughout the experiment. The following measurements were performed:

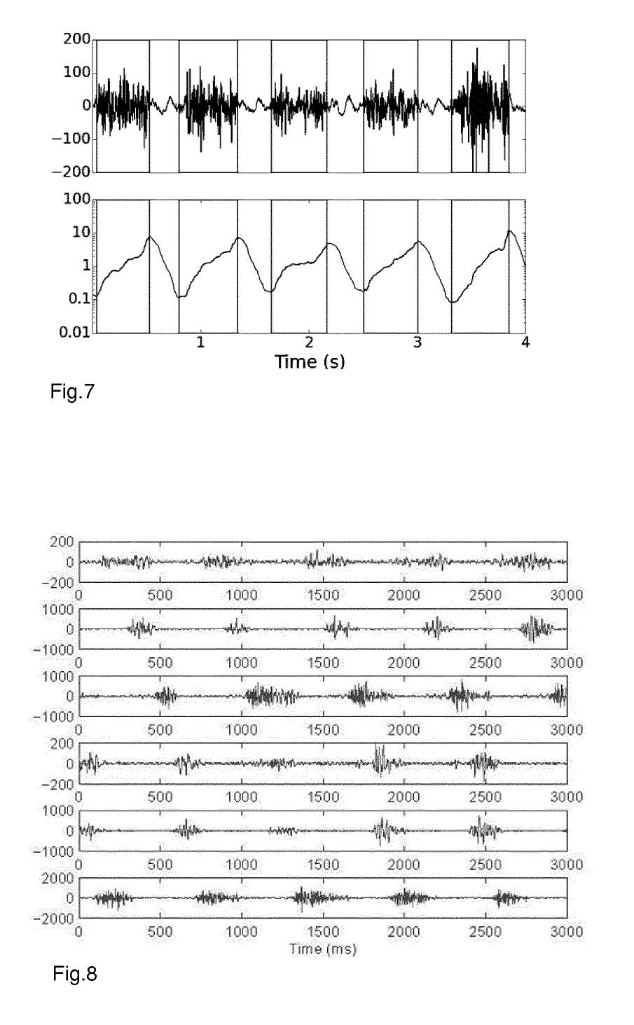

1. EMG signals were obtained from right and left rectus femoris (RRF, LRF), right and left vastus lateralis (RVL, LVL), and right and left semitendinosus (RST, LST) muscles using bipolar surface electrodes (BlueSensor, AMBU, Copenhagen, Denmark) connected to an eight-channel EMG recorder (Muscle Tester 6000, Megawin, Kuopio, Finland) at a sampling rate of 1000 Hz. The skin was shaved and cleaned with a 0.5 mg/mL solution of chlorhexidine (Fresenius Kabi, Bad Homburg, Germany), and was allowed to air dry for 1 min before application of electrodes. EMG cross-talk was minimized by placing the electrodes within the border of the specific muscle, and with a center-to-center inter-electrode distance of 22 mm. An example of the recorded signals can be seen in FIG. 7.

2. Blood lactate concentration was determined approximately every minute with LactatePro2 (Arkray Europe B.V., Amstelveen, the Netherlands) using blood collected from subject's fingertips.

3. VO₂ (oxygen uptake) measurements were made every 10 seconds throughout the tests with Jaeger Oxycon Pro (CareFusion, San Diego, Calif., USA). Due to equipment malfunctions, measurements were lost for subjects 1, 3, and 9.

Electronic noise and motion artifacts were removed from the sEMG signals using Butterworth filters. For the electronic noise and other high frequency content, a 10^{th} order 400 Hz low-pass filter (450 Hz stop band with at least −60 dB attenuation) was used. Similarly, a 10th order 20 Hz high-pass filter (10 Hz stop band with at least −60 dB attenuation) was used to remove motion artifacts.

To ensure signal amplitudes were on similar scale for all subjects and muscles, EMG signals were normalized using the root-mean-square (RMS) amplitude of the first 100 pedal revolutions. There are more complex and precise methods for EMG normalization. However, described normalization was chosen for an eventual use in consumer grade equipment.

The physiological parameters (lactate concentration and oxygen uptake) were interpolated where needed using Hermite cubic splines with Catmull-Rom tangents.

A feature extraction for regression models was then performed. The most significant timing events for EMG signals are the moments where muscle changes from an active state to passive or vice versa. The following algorithm was used for the purpose of detecting such events. For each time moment t, the ratio of variability R(t) in a window of a fixed length T before that moment versus a similar window after that moment was calculated. In other words, assuming a signal S(t),

$$R ( t ) = \frac{{\sum }_{τ = t - T}^{t} \vert S ( τ ) - S ( τ - 1 ) \vert}{{\sum }_{τ = t}^{t + T} \vert S ( τ ) - S ( τ - 1 ) \vert} \qquad ( 1 )$$

When a muscle is active, the corresponding EMG signal is a lot more variable, which can be expressed in larger absolute values of the derivative of the signal, while they stay relatively low when the muscle is inactive. It then follows that during a transition from passive to active state, R(t) will reach its local minimum value, and similarly it will reach its local maximum value when the opposite transition occurs. This behavior is illustrated in FIG. 7 showing an EMG signal (ordinate) as a function of time (abscissa) in the top diagram and the corresponding R(t) function (ordinate) in the bottom diagram as a function of time (abscissa). A correspondence between muscle activity and the extrema of R(t) can be seen in FIG. 7. For this investigation, the size of the window was selected to be T=256 samples (256 ms, roughly a third of a pedal cycle), as it provided the best event detection results.

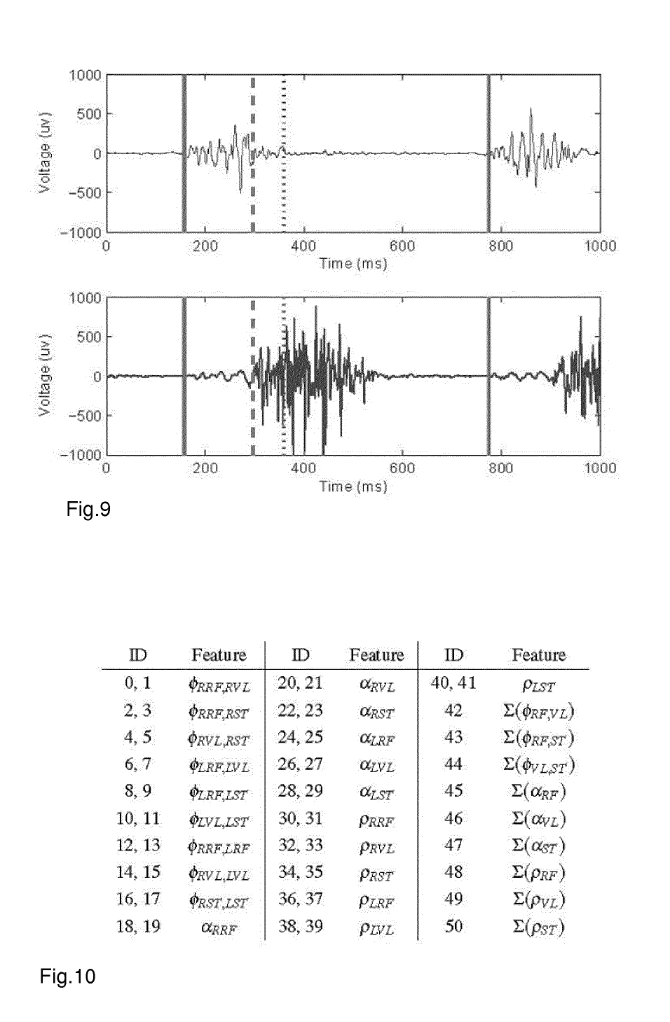

For the i-th revolution of bicycle pedals there are 12 corresponding EMG timing events, marking activation time A*(i) and deactivation time D*(i) of each of the six muscles that the readings were taken from. A typical structure of the activation pattern for these muscles can be seen in FIG. 8, and the definitions of timing events can be seen in FIG. 9.

FIG. 8 shows the structure of the EMG signals (ordinate) as a function of time (abscissa) from top to bottom for the RRF, RVL, RST, LRF, LVL, and LST. It can be seen that these muscles fire sequentially during a single cycle of pedaling. FIG. 9 shows a visual guide to timing event definitions using signals (ordinate) from RRF and RVL as functions of time (abscissa). The solid lines correspond to activations of RRF A_{RRF}(i) and A_{RRF}(i+1), the dashed line corresponds to activation of RVL A_{RVL}(i), and the dotted line corresponds to deactivation of RRF D_{RRF}(i).

During a single cycle, the sequence of muscles firing is stable, therefore, the time interval between two consecutive activations of rectus femoris of the right leg was considered to be the length of one revolution of pedals, and all other time periods were calculated as fractions of this baseline time length. Namely, for two muscles X and Y, the phase shift in cycle i ϕ_{X, Y}(i) is defined as:

$${φ}_{X , Y} ( i ) = \frac{{A}_{Y} ( i ) - {A}_{X} ( i )}{{A}_{RRF} ( i + 1 ) - {A}_{RRF} ( i )} \qquad ( 2 )$$

For a muscle X, the active-time percentage αₓ is defined as:

$${α}_{X} ( i ) = \frac{{D}_{X} ( i ) - {A}_{X} ( i )}{{A}_{RRF} ( i + 1 ) - {A}_{RRF} ( i )} \qquad ( 3 )$$

The root-mean-square amplitude ρₓ(i) for the i-th cycle of the EMG signal from a muscle X was calculated over the window between Aₓ(i) and Dₓ(i):

$${ρ}_{X} ( i ) = \sqrt{{\sum }_{t = {A}_{X} ( i )}^{{D}_{X} ( i )} {s}^{2} ( t )} \qquad ( 4 )$$

All aforementioned features were considered over sliding windows of N pedal revolutions, where their arithmetic mean E[*] and standard deviation σ[*] was calculated:

$$\begin{matrix}E [ * ] ( i ) = \frac{1}{N} {\sum }_{j = i - N + 1}^{i} * ( i ) & ( 5 ) \\ σ [ * ] ( i ) = \sqrt{\frac{1}{N - 1} {\sum }_{j = i - N + 1}^{i} (* {( j ) - E [ * ] ( i ) )}^{2}} & ( 6 )\end{matrix}$$

In addition, the symmetry E(*) was calculated for each applicable feature by taking the corresponding means for the right and the left leg (E[*_{R}] and E[*_{L}]), and then finding the absolute difference between them:

Σ(*)=|E[*_{R}]−E[*_{L}] (7)

When E[*_{R}]=E[*_{L}], the symmetry Σ(*) is 0, otherwise it is a positive number.

In total, the set of input features or parameters contained 51 different measures or features, all derived directly from time-domain data. These features are listed in the table of FIG. 10 showing a complete list of features used in regression models. Two IDs assigned to the same feature correspond to the mean (E[*], first ID) and the standard deviation (σ[*], second ID) over the window of N last measurements. Feature extraction was performed using a custom-written Java 7 (Oracle, Redwood City, Calif., USA) application.

Linear models with Tikhonov's (ridge) regularization, as well as random forests were used to design the regression models to predict blood lactate concentration or oxygen uptake.

Test set (10% of data points for linear models) and out-of-bag (for random forests) data were used to assess the performance of these regression models by calculating the coefficient of determination.

$${R}^{2} = 1 - \frac{{\sum }_{i} {( {Y}_{i} - {\hat{Y}}_{i} )}^{2}}{{\sum }_{i} {( {Y}_{i} - E [ Y ] )}^{2}} \qquad ( 8 )$$

Here Yᵢ are the actual values, E[Y] is the mean value of the test data, and Ŷᵢ are the predictions made by the model.

R² is a normalized measure of regression quality, where 1 signifies the perfect regression model (predicted values are exactly equal to the actual values), and 0 signifies the naive regression model (predicted values are the mean of the actual values). To ensure a good estimate of R², 10-fold cross-validation was used for linear models, and random forests were rebuilt 10 times using different initial seeds.

An important consideration when testing regression models is contribution of different inputs to the generated output, i.e., whether some particular input variable can be safely discarded without significant loss to the predictive power. For that purpose, a sequential backward elimination algorithm was used, with R² estimate from out-of-bag data as the criterion.

Regression models, as well as the input variable pruning algorithm, were implemented using Matlab R2012b (Mathworks, Natick, Mass., USA).

For the linear ridge regression, the best shrinkage parameter was selected experimentally by trying out different values and selecting the ones that gave the best R² values when using 10-fold cross-validation. For the random forests, common parameter choices were used: 100 decision trees, one third of input features considered at each split, and minimum node size set at 5.

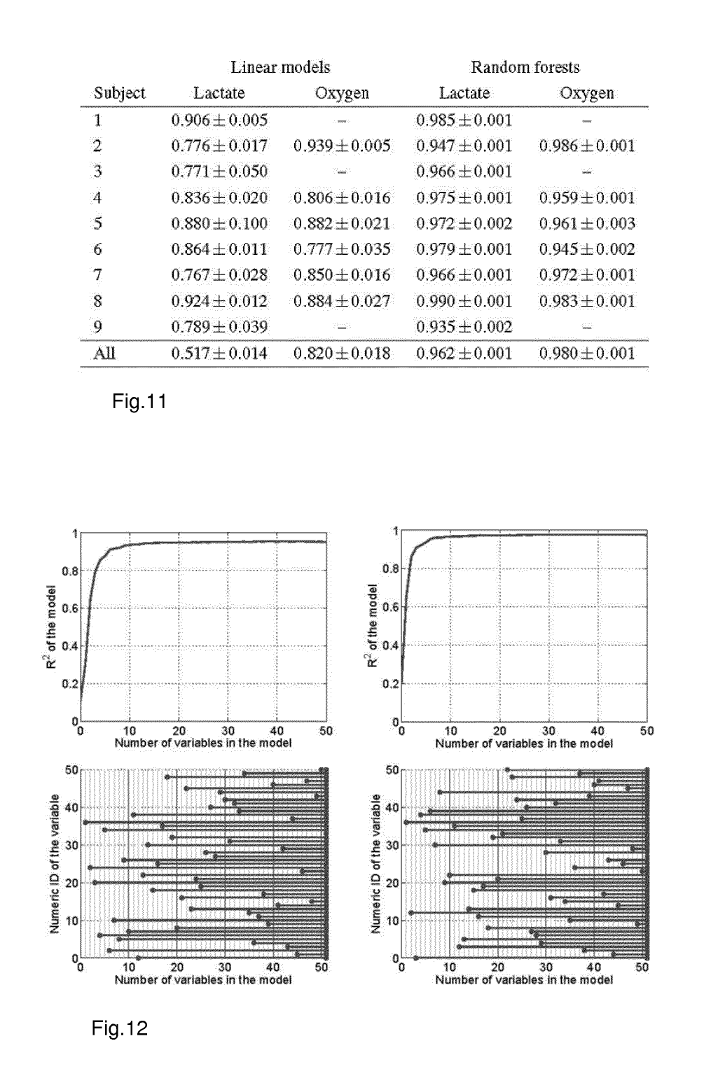

The results for the linear and random forest regression using the full variable set are presented in the table of FIG. 11 showing R² estimates from out-of-bag/test sets for individual data sets as well as combined data.

These results show that even linear models provided very good (R²>0:76) prediction quality for both blood lactate concentration and oxygen uptake, although in the former case, the accuracy deteriorated a lot when the data sets were combined. However, random forest regression provided excellent prediction accuracy (R²>0:93) and deterioration from combining multiple data sets was not observed.

FIG. 12 shows the results of the abovementioned variable pruning procedure, or discarding of input variables. The top diagrams shows the relationship between the number of input variables (abscissa) and the R² (ordinate) of the random forest model, where the left diagram predict blood lactate concentration and the right diagram predict oxygen uptake. The bottom diagrams are the respective inclusion matrices for input variables used in the models above, showing the number of variables in the model (abscissa) for respective number ID of the variable (ordinate).

It can be construed from FIG. 12 that very good prediction accuracy (R²>0:9 can be obtained with as few as 7 variables for blood lactate concentration and 4 variables for oxygen uptake. For blood lactate concentration, these variables are:
mean RMS amplitude of left rectus femoris (E[ρ_{LRF}],36);
mean active time of left rectus femoris (E[α_{LRF}],24):
mean active time of right vastus lateralis (E[α_{RTL}],20);
mean phase shift between left rectus femoris and left vastus lateralis (E└ϕ_{LRF,LVL}┘,6);
mean RMS amplitude of right semitendinosus(E[ρ_{RST}],34);
mean phase shift between right rectus femoris and right semitendinosus (E└ϕ_{RRF,RST}┘,2);
mean phase shift between left vastus lateralis and left semitendinosus (E└ϕ_{LVL,LST}┘,10).

For oxygen uptake, these variables are:
mean RMS amplitude of left rectus femoris(E[α_{LRF}],36);
mean phase shift between right and left rectus femoris (E└ϕ_{RRF,LRF}┘,12);
mean phase shift between right rectus femoris and right vastus lateralis (E└ϕ_{RRF,RVL}┘, 0);
mean RMS amplitude of left vastus lateralis (E[α_{LVL}],38).

The results outlined above are considerably better than what is typically achieved using the spectral properties from the frequency domain of EMG signals. The increased accuracy of linear models is particularly surprising, and indicates that there are fundamental changes in muscle employment strategies and resultant kinematics as the cyclist fatigues, blood lactate concentration rises, and more oxygen is consumed. It is contemplated that a similar result and conclusion can be achieved for other sports and exercises. Even more importantly, it has been shown that even a very small subset of the defined time-domain variables is sufficient to produce random forest models of high accuracy. It is also contemplated that a similar result and conclusion can be achieved for other sports and exercises.

It can also be construed from the above study that a model can be formed for determining a measure based on a limited number of input parameters, and that the same parameters can be used for different persons. This means that a minimal adaption of the model to a user is required.

A significant result is that time-domain features, which typically are simpler to compute than frequency-domain features, can be used for accurate determination of the blood lactate concentration and the oxygen uptake. This allows for applications that are easy to carry

Another significant result is that models predicting blood lactate concentration appear to rely on interactions between front and back thigh muscles, while models predicting oxygen uptake appears to rely exclusively on front muscles, but also take into account the difference between the two legs.

The proposed technology allows for an estimate of physiological parameters relating to fatigue without relying on MPF or other spectrum derived measures. Instead, the timing of different events in parallel sEMG signals from a few different muscles is used.

From the description above follows that, although an embodiment of the invention has been described and shown, the invention is not restricted thereto, but may also be embodied in other ways within the scope of the subject-matter defined in the general description and the following claims. Throughout these specifications, a mean is understood to encompass an arithmetic mean.

1. A method for determining a measure indicating the fatigue of a person, the method comprises:
obtaining (102) a plurality of simultaneously recorded signals, wherein each signal is associated with a muscle of the person and indicates electrical activity of the associated muscle in the time domain;
determining (104) a set of points in time from each signal, wherein each point in time indicates a change in the state of the associated muscle; and
determining (106) the measure indicating the fatigue of the person based on the sets of points in time for the plurality of simultaneously recorded signals.

2. The method according to claim 1, wherein the set of points in time is composed of activation times and deactivation times, wherein each activation time indicates the start of an active state of the associated muscle, and each deactivation time indicates the start of an inactive state of the associated muscle.

3. The method according to claim 2, wherein the method further comprises:
determining (108) from each signal an indication of a change in activity over time of the associated muscle, and wherein the set of points in time for the associated muscle are derived from the indication of the change in activity over time.

4. The method according to claim 2, wherein determining (106) the measure indicating the fatigue comprises:
determining (112) one or more mean root-mean square values, wherein
each of these root-mean square values is determined (116) as the mean of a plurality of root-mean square values determined from a single signal of the plurality of simultaneously recorded signals; and
each of these root-mean square value is determined (114) based on the root-mean square of the amplitude for a time period of the single signal, wherein
each time period is located between an activation time and a directly following activation time of the single signal, and wherein
the measure indicating the fatigue is determined (106) based on the determined one or more mean root-mean square values.

5. The method according to claim 2, wherein determining the measure indicating the fatigue comprises:
determining (118) one or more mean phase shifts, wherein
each of these mean phase shifts is determined (124) as the mean of a plurality of phase shifts determined from a pair of signals of the plurality of simultaneously recorded signals, and
each of these phase shifts is determined (120) based on a first time interval between an activation time of a signal of the pair of signals and a directly following activation time of the other signal of the pair of signals, and
wherein the measure indicating the fatigue is determined based on the determined one or more mean phase shifts.

6. The method according to claim 5, wherein, for each mean phase shift, the plurality of phase shifts is further determined from a first additional signal of the plurality of simultaneously recorded signals, and
each first time interval is normalized (122) by a second time interval between two consecutive activation times of the first additional signal and covering at least a portion of the first time interval.

7. The method according to claim 2, wherein determining (106) the measure indicating the fatigue comprises:
determining (126) one or more mean active-time intervals, wherein
each of these mean active-time intervals is determined (132) as the mean of a plurality of active-time intervals determined from a single signal of the plurality of simultaneously recorded signals, and
each of these active-time intervals is determined (128) based on a third time interval between an activation time and a directly following deactivation time, and
wherein the measure indicating the fatigue is determined (106) based on the determined one or more mean active-time intervals.

8. The method according to claim 7, wherein, for each mean active-time interval, the plurality of active-time intervals is further determined from a second additional signal of the plurality of simultaneously recorded signals, and
each third time interval is normalized (130) by a fourth time interval between two consecutive activation times of the second additional signal and covering at least a portion of the third time interval.

9. A system for determining a measure indicating the fatigue of a person, the system comprising one or more detectors for simultaneously recording signals indicating electrical muscle activity in the time domain, a processor for executing program instructions, and a non-volatile memory comprising program instructions configured to, when executed by the processor, cause the system to:
obtain a plurality of simultaneously recorded signals from the one or more detectors, wherein each signal is associated with a muscle of the person and indicates the electrical activity of the associated muscle in the time domain;
determine a set of points in time from each signal, wherein each point in time indicates a change in the state of the associated muscle; and
determine the measure indicating the fatigue of the person based on the set of points in time from the plurality of simultaneously recorded signals.

10. A computer program product for use in a system for determining a measure indicating the fatigue of a person, the system comprising one or more detectors for simultaneously recording signals indicating electrical muscle activity in the time domain, and a processor for executing program instructions, wherein the computer program product comprises program instructions configured to, when executed by the processor, cause the system to:
obtain a plurality of simultaneously recorded signals from the one or more detectors, wherein each signal is associated with a muscle of the person and indicates the electrical activity of the associated muscle in the time domain;
determine a set of points in time from each signal, wherein each point in time indicates a change in the state of the associated muscle; and
determine the measure indicating the fatigue of the person based on the set of points in time from the plurality of simultaneously recorded signals.
